(19) United States
(12) Patent Application Publication
(10) Pub. No.: US 2002/0013574 A1
(43) Pub. Date: Jan. 31, 2002

(54) METHOD AND ARRANGEMENT FOR PHACOEMULSIFICATION

(76) Inventors: JENS ELBRECHT, JENA (DE); BERLIND KALVE, JENA (DE); ECKHARD SCHROEDER, ECKENTAL (DE)

Correspondence Address: REED SMITH LLP, 375 PARK AVENUE, NEW YORK, NY, 10152, US

(21) Appl. No.: 09/067,179

(22) Filed: Apr. 27, 1998

(30) Foreign Application Priority Data
Apr. 30, 1997 (DE) 19718139.2

(51) Int. Cl. A61B 18/22

(52) U.S. Cl. 606/5; 606/15

(57) ABSTRACT

A method and arrangements for phacoemulsification are disclosed in which the biological tissue of the eye lens is ablated by the application of energy in the form of pulsed laser radiation and the occurring ablation product is removed from the treatment site by suction. According to the invention, the object is met by a method of the type mentioned above in that the application of energy in the course of the comminution of nucleus parts is influenced by changing the pulse train and/or the intensity of the laser radiation. A change in the pulse train should advantageously be provided in such a way that an interruption in the laser radiation of at least one pulse length is carried out after every series of pulses at a constant pulse frequency.

### BACKGROUND OF THE INVENTION

1. Field of the Invention

The invention is directed to a method for phacoemulsification in which the biological tissue of the lens of the eye is ablated through the application of energy in the form of pulsed laser radiation and the occurring ablation product is removed by suction from the treatment site. The invention is further directed to arrangements for carrying out this method in which a comminution of the biological tissue of the eye lens is provided by the action of pulsed laser radiation, with a laser, with a driving circuit for the laser, and with at least one light waveguide which transmits the laser radiation to the treatment site and radiates it into the tissue at that location.

2. Description of the Related Art

Phacoemulsification in which a cannula is inserted into the eye in order to suction off the cataractous lens nucleus has been known since its inception in 1967. While nucleus fragmentation is carried out by ultrasonic shocks in conventional method variants, laser phacoemulsification makes use of a pulsed laser beam for this purpose.

The effect of the laser beam can be utilized indirectly by way of the action of shock waves as is suggested in the “laser system for intraocular tissue removal” described in U.S. Pat. No. 4,694,828. Alternatively, however, the laser beam can also be aimed directly on the tissue to be comminuted. For the latter method, a laser wavelength with a very shallow penetration depth into aqueous material of the interior of the eye must be selected. Suitable wavelengths for this purpose are the low UV or infrared range. In this connection, the use of Er:YAG lasers with a wavelength of λ=2.9 μm has proven successful because, in theory, the penetration depth in water amounts to only a few micrometers. The disruptive effect only takes place directly at the end of the light waveguide and has a penetration depth into the material of less than 1 mm. An effect which occurs in this connection is an explosive evaporation of the aqueous material directly in front of the light exit or outlet surface of the light waveguide, resulting in the formation of a cavitation bubble which collapses immediately with each individual laser shot.

For effective comminution of the harder nucleus parts, a pulse frequency of the laser radiation is used which, over the course of the historical development of laser phacoemulsification, has moved from an initial several hertz to 100 Hz and more at the present time. While faster comminution is carried out in this way, the following problem results in a disadvantageous manner: When the light waveguide tip or fiber tip is pushed into the nucleus material during the laser shot procedure, the high pulse train can cause cavitation chambers to be formed in the material which do not then collapse and cannot be completely filled with rinsing fluid before the arrival of the next laser pulse. This can have the result that the chamber which has already formed can become even larger. This can lead to shot grooves or shot channels with a depth of 2, 3 or even 4 mm, which can lead to rupturing of the capsular bag.

### OBJECT AND SUMMARY OF THE INTENTION

The primary object of the invention is to further develop the method mentioned above for laser phacoemulsification and the arrangements known up to this point for carrying out the method such that the risks involved in deep penetrative action of the laser radiation are avoided.

According to the invention, this object is met by a method of the type mentioned above in that the application of energy in the course of comminuting the nucleus parts is influenced by changing the pulse train and/or the intensity of the laser radiation. A change in the pulse train should advantageously be provided in such a way that an interruption in the laser radiation of at least one pulse length is carried out after every series of pulses at a constant pulse frequency.

This results in the substantial advantage that a cavitation space formed by the action of laser radiation can fill up with water in the intervals which occur repetitively after a series of laser pulses and in which the laser radiation does not act on the tissue. The ratio of the acting period to the interruption period can be selected in such a way that the cavitation space can fill up with fluid or can be collapsed by suction before additional energy in the form of laser radiation is introduced into the tissue by a further series of pulses.

If the end of the phaco tip penetrates into the nucleus material of the lens of the eye during ablation, it is surrounded by the gel-like nucleus material so that it is no longer possible for the rinsing fluid, generally water, to flow into the cavitation space formed by the action of the laser, and the risk of unwanted deep penetrative action described above occurs when the next series of pulses enters the same cavitation space which has not yet collapsed and is not yet filled with fluid.

However, the brief interruption of the pulse train enables timely evacuation of the cavitation space, so that this cavitation space cannot grow larger and so that the above-described risk is excluded. After the evacuation of the cavitation space, the gel-like nucleus material immediately surrounds the tip again and the water located in the cavitation space ensures that the ablation can only be carried out in the immediate vicinity of the end of the tip. The method according to the invention thus prevents excessively deep penetrative action of the laser radiation in the described manner by ensuring that the cavitation space can be evacuated with sufficient speed and can subsequently be filled with water again quickly.

In a particularly advantageous configuration of the method according to the invention, a value is selected for the constant pulse frequency in the range of 40 Hz to 100 Hz and a value is selected for the ratio of the duration of a pulse series to an interruption in the range of 10:1 to 1:1. In this way, an optimum evacuation of the ablation product can be achieved and an excessively deep penetrative action which can lead to the described unwanted secondary effects is successfully prevented.

An alternative variant of the method according to the invention can consist in that the pulse train is influenced in that the pulse frequency is alternately increased and reduced. In so doing, a sufficiently fast evacuation of the cavitation space is achieved and damaging secondary effects are prevented also because of the reduced application of energy during the reduced pulse frequency.

In a further preferred development of the invention, the application of energy is varied depending on the optical characteristics of the material at the treatment site. In accordance with a development of the invention, the return reflection of the laser radiation from the light outlet surface of a light waveguide serving to transmit the laser radiation to the treatment site can be evaluated and changed depending on this return reflection of the applied energy.

Thus, by using the return reflection of the laser radiation, e.g., of the red diode light, it is possible to monitor during the shot sequence whether gas is present in front of the light outlet surface, i.e., at the effective location of the laser radiation, and accordingly at the treatment site. If this signal indicates that gas is located in front of this end surface, the laser radiation is interrupted immediately or after a preselectable time period using this signal until the consistency of the material in front of the light outlet surface changes again and the presence of fluid or tissue (lens material) at this location can be deduced from the return reflection. The resulting change in the return reflection signal is then used to initiate the release of the laser radiation.

This leads to the important advantage that the ratio of the duration of a pulse series to the duration of the interruption need not be preset in a fixed manner, but rather an interruption can be carried out precisely when required by the given factors at the treatment site. In this way, an amount of energy can be applied which is suited to the given situation at the treatment site.

In a preferred further development of the method according to the invention, the radiating direction of the laser radiation in the tissue is varied depending on physical data of the tissue at the treatment site. In this way, in a constructional variant of the invention, the radiation direction can be varied depending on the optical characteristics of the material at the treatment site. Also, for this purpose the return reflection of the laser radiation which is directed from the inside of the laser outlet surface into the light waveguide can be evaluated and the radiation direction can be changed in dependence thereon, wherein the change in the radiating direction is carried out when the material located at the treatment site passes into the gaseous state.

If the light outlet surface of the light waveguide is inclined relative to the center axis of the light waveguide, which results in radiation of the laser light lateral to the center axis, the change in the radiation direction can also be carried out, for example, in that the light waveguide is rotated about its center axis. The rotation of the light waveguide about its center axis can be initiated after every pulse series or also as the outcome of the evaluation of the return reflection from the light outlet surface of the light waveguide.

As was already described above, the return reflection from the light outlet surface supplies information about the consistency of the substance in front of the light outlet surface. If it can be deduced from the return reflection that gas is present in front of the outlet surface, a rotation of the light waveguide is effected about its center axis. As a result, the laser radiation is no longer directed into the cavitation space filled with gas which could lead to disadvantageous deep penetrative action.

In a further development of the invention, the change in the radiation direction is carried out in dependence on the pressure at the treatment site. For this purpose, for example, the pressure increase related to the propagation of gas in the cavitation space is made use of for the purpose of developing on the surface arranged at the end of the tip a force component which causes a deflection of the light waveguide lateral to the center axis and, accordingly, a change in the radiation direction of the laser radiation in the tissue. This surface, which makes it possible to obtain a force component from the increasing pressure which can act in a determined direction given by the inclination of the surface, can be the light outlet surface of the light waveguide that is inclined relative to its center axis. However, it is also possible to provide separate surfaces with a corresponding inclination at the end of the light waveguide.

The signal containing information about the pressure at the treatment site can also conceivably be used in a manner similar to the procedure described above to influence the application of energy for the comminution of the nucleus parts by changing the pulse train and/or the intensity of the laser radiation.

The invention is further directed to an arrangement for phacoemulsification with a laser, with a driving circuit for the laser, and with at least one light waveguide which transmits the laser radiation to the treatment site and radiates it into the tissue at the treatment site, wherein a device for influencing the pulse train and/or the intensity of the laser radiation is provided. In a development of the arrangement according to the invention, a controllable optical interrupter which is arranged in the laser beam path can be provided as a device for influencing the pulse train.

Further, the driving circuit for the laser can communicate with a control input of the optical interrupter via a pulse counter, wherein the pulse counter has a reference value input and can be adjusted by this reference value input in such a way that a switching signal is supplied to the optical interrupter after every cycle of a set reference number of pulses to be counted, wherein the switching signal causes a blocking of the passage of the laser radiation for the duration of at least one pulse length.

Accordingly, it is possible after every cycle of a predetermined series of pulses which is recorded by the pulse counter to cause the optical interrupter to be switched on again and interrupt the laser beam path.

However, it can also be advantageously provided that the reference value input of the pulse counter is connected to the signal output of an optoelectronic receiver whose receiving direction is directed to the inside of the light outlet surface of the light waveguide. Thus, it is also possible in this way to apply an amount of energy suitable to the respective situation at the treatment site.

A further very advantageous configuration of the arrangement for laser phacoemulsification, in which the occurring ablation product is removed by suction through a mouth opening of a suction tube, consists in that the light waveguide or light waveguides, insofar as more than one is provided, is or are arranged such that at least a portion thereof extends in the suction tube and the laser radiation is directed out of the mouth opening of the suction tube to the treatment site, wherein the end of the light waveguide on the light exit side is enclosed annularly by the suction tube cross section.

The occurring ablation product is accordingly removed by suction around the end of the light waveguide or around the ends of the light waveguides. The material to be suctioned thus reaches the mouth opening directly at its site of origin without its removal being hampered by unfavorable flow conditions.

It can be provided in an advantageous manner that there is only one light waveguide, wherein the cross section of the light waveguide and the cross section of the suction tube are round and both cross sections are arranged eccentrically relative to one another with respect to their center of curvature. As a result of this eccentric arrangement of the light waveguide in the round cross section of the suction tube, the annular gap of the mouth opening varies in width at its circumference. This has the advantageous result that larger ablation products can flow without hindrance into the opening with the larger width and can be removed through the suction tube.

In a special construction, the cross-sectional surface of the suction tube is not constant over its length, but can widen, for example, starting at the mouth opening, into the suction tube so as to result in a conical end of the suction tube at the mouth.

In further configurations of the arrangement according to the invention, the inlet opening of the suction tube is inclined relative to the center axis of the light waveguide by an angle a α≠90° and the light outlet surface at the light waveguide is inclined relative to the center axis of the light waveguide by an angle β≠90°. The inclination of the light outlet surface results in a deflection of the laser radiation when exiting from the light waveguide, wherein the laser radiation can be advantageously oriented in such a way that it impinges at least partially on the inner wall of the suction tube, is reflected from the latter, and strikes the tissue to be destroyed. Moreover, the comminution of tissue already located in the mouth opening is made possible in this way.

In a very preferable configuration of the arrangement according to the invention, the light waveguide is arranged within the suction tube so as to be rotatable about its center axis and is connected with a rotary drive. This advantageously results in that the radiation direction of the laser radiation into the tissue can be changed by the rotation of the light waveguide about its center axis.

This change can be carried out when there is a risk of increased depth action due to the influence of the laser radiation in a cavitation space that is filled with gas. The rotation of the light waveguide about its center axis can be initiated manually by means of the rotary drive, but it is also possible for the rotary drive to have a controlling device linked with the signal output of an optoelectronic receiver which is directed to the inner side of the light outlet surface for the laser radiation at the light waveguide with respect to its reception direction. This arrangement also makes it possible to make use of the return reflection from the light waveguide which was described above and to initiate the rotation of the light waveguide, and accordingly the change in the radiation direction of the laser radiation in the tissue, depending on the presence of gas in front of the outlet surface.

Further, it is possible to provide a plurality of light waveguides, to construct the light outlet surfaces of the individual light waveguides with different inclinations, and thus to achieve a focussing of the laser radiation introduced into the tissue from the individual light waveguides. This has the advantage that while the comminution of the tissue is carried out in the focus of this laser radiation or in its immediate neighborhood, this is no longer the case in the further course of the laser radiation because the laser radiation diverges and a deep penetrative action is consequently no longer possible.

In a further variant of the arrangement, the individual light waveguides are alternately acted upon one after the other by laser radiation and this laser radiation is alternately introduced into the tissue from the individual light waveguides. The outlet ends of these alternately controlled light waveguides can be oriented in such a way that the application of energy is carried out at least partially in other tissue parts.

Further solutions for preventing deeper shot channels are possible, for example, through the arrangement of a protective shield for limiting the range of the laser radiation. In this case, the laser radiation is scattered at a distance from the light outlet surface at the protective shield, wherein this distance is predetermined by the arrangement of the protective shield, and can accordingly not cause further ablation or formation of shot channels.

The light waveguide can be constructed as a light-conducting fiber or as a waveguide or hollow conductor.

It should also be noted that there are possibilities for forming the light outlet surface of a light-conducting fiber as an optical functional surface, for example, as a concave surface which acts like a scattering lens, as a convex surface which focusses the laser light with a short focal length and in which the laser light diverges sharply after traversing the focal point, as a conical tip with a very acute angle so that the laser light exits on the entire length of the conical slice or cut end and accordingly radiates more broadly, or, finally, also by means of a special cut angle by means of which the light cone of the exiting laser beam is enlarged.

There follows a description of some of the arrangements, according to the invention, by means of which it is also possible to utilize the method according to the invention at the same time.

### BRIEF DESCRIPTION OF THE DRAWINGS

In the drawings:

FIG. 1 is a schematic view of the arrangement according to the invention with optical interrupter;

FIG. 2 is also a schematic view of the arrangement according to the invention with an optical interrupter and photoelectric receiver;

FIG. 3 shows a first constructional variant of the phaco tip;

FIG. 4 shows a side view A from FIG. 3;

FIG. 5 shows a second constructional variant of the phaco tip;

FIG. 6 illustrates a third constructional variant of the phaco tip;

FIG. 7 illustrates a fourth constructional variant of the phaco tip; and

FIG. 8 shows a fifth constructional variant of the phaco tip.

### DESCRIPTION OF THE PREFERRED EMBODIMENTS

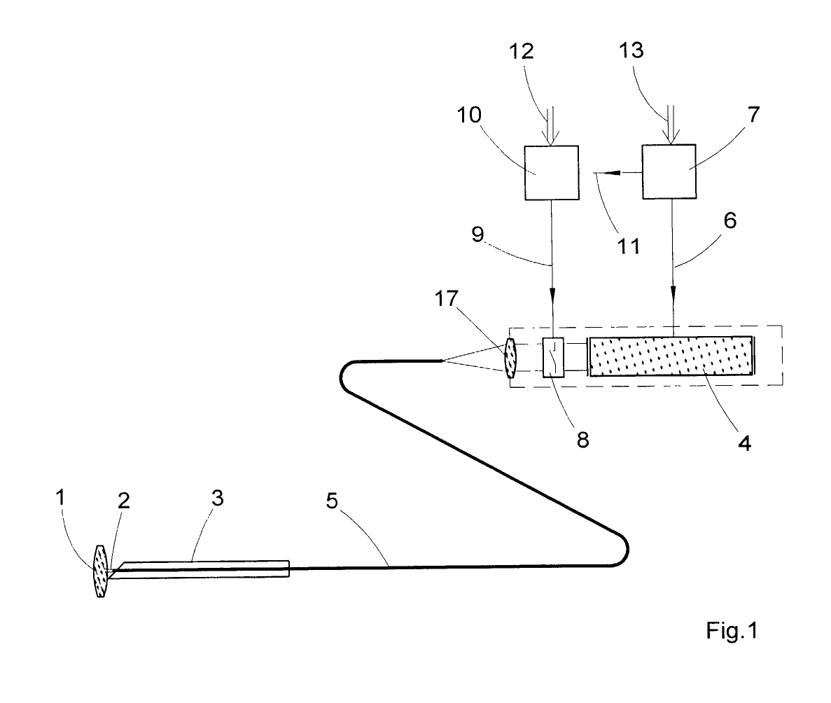

FIG. 1 shows a symbolic representation of a lens 1 of an eye in which energy in the form of laser radiation 2 is introduced for the purpose of phacoemulsification. For this purpose, the phaco tip 3 which is constructed as a handle serves for manual orientation of the laser radiation 2 on the treatment site at the eye lens 1. The ground end of the phaco tip 3 is first introduced into the anterior chamber of the eye, the irrigation and aspiration according to medical instrument technique is then switched on and the laser 4 is put into operation. While changing the orientation of the phaco tip 3, the nucleus of the eye lens 1 is comminuted in the capsular bag by the action of the laser radiation 2 and the fragments are removed by suction. The laser radiation 2 is generated in the laser 4 which is arranged so as to be separated locally from the phaco tip 3 and is transmitted via the light-conducting fiber 5 from the laser 4 to the phaco tip 3.

The laser 4 is connected, via the signal path 6, with a driving circuit 7 which, in addition to starting operation, also makes it possible, for example, to adjust the pulse frequency. An optical interrupter 8 is arranged in the beam path of the laser radiation 2 between the laser 4 and the end of the light-conducting fiber 5 on the light source side.

The optical interrupter 8 is connected, via the signal path 9, with control electronics 10 which are connected, in turn, via the signal path 11, with the driving circuit 7 for the laser 4.

Prior to treatment, a presetting of the desired pulse frequency, for example, 80 Hz, is carried out by means of the driving circuit 7 and the laser is put into operation. With this shot sequence frequency of 80 Hz, the nucleus material is comminuted and the phaco tip is inserted into the nucleus material according to the rate at which comminution proceeds.

At this pulse frequency of 80 Hz, the comminution proceeds relatively quickly. In so doing, there is a risk of the formation of cavitation chambers that can not collapse sufficiently or that can not be filled with fluid quickly enough by irrigation (not shown in the drawing).

In order to prevent consequent formation of shot channels with a length of 2 mm or more when the next fast series of shots is directed into a gas-filled cavitation chamber, the laser beam path 2 is blocked by means of the optical interrupter 8 after a predetermined quantity of pulses. For this purpose, the control electronics 10 contain a pulse counter which receives the pulses emitted by the laser 4 as a counting sequence via the signal path 11. The control electronics 10 can be preset via the input 12 in such a way that the optical interrupter 8 is actuated after a preselectable pulse number whose delivery via the signal path 11 is monitored by the control electronics 10, and the laser beam path 2 is interrupted by switching on the optical interrupter 8. For this purpose, the quantity of pulses to be emitted by the laser 4 before the first interruption is set via the input 12 and the duration of the interruption is also predetermined. The duration of the interruption can be equivalent to a pulse number. In this way, the ratio of the duration of an emitted pulse train to the duration of the interruption can be adjusted equivalent to a number of pulses. This ratio can be 3 to 1, for example. This can be realized in such a way, for example, that after six pulses which are emitted at a frequency of 80 Hz, two additional pulses are suppressed.

After the expiration of the blocking period, the laser beam path is released by switching off the optical interrupter 8 and the laser radiation 2 impinges on the nucleus material with a further series of shots which corresponds to the quantity of pulse numbers predetermined by the input 13 via the driving circuit 7. After this pulse number is recorded by the control electronics 10, the optical interrupter 8 is switched on again for the duration of a predetermined pulse train.

This process is repeated at intervals so that a pause period follows every series of shots at the treatment site. The continuously recurring pause periods make it possible for the cavitation spaces to collapse and fill with fluid, so that the formation of shot channels with a length of several millimeters is prevented.

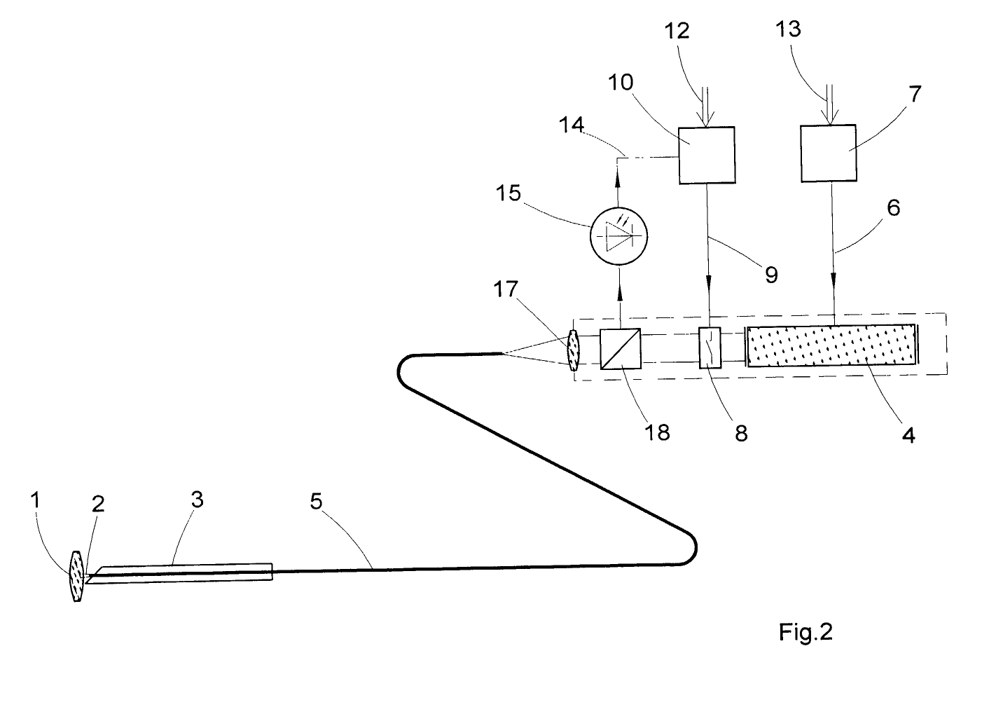

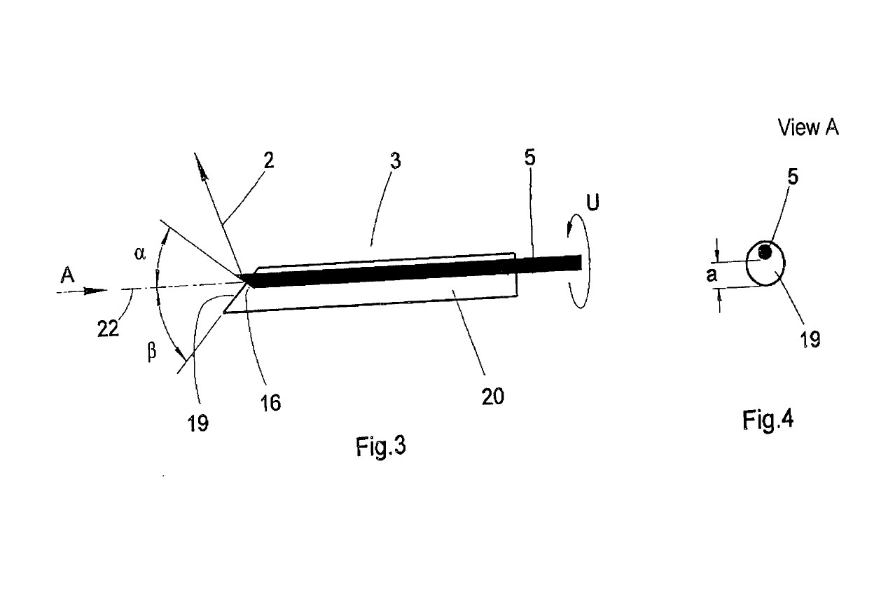

FIG. 2 shows a constructional variant of the arrangement described above in which the control electronics 10 are linked with the output of an optoelectronic receiver 15 via a signal path 14. The optoelectronic receiver 15 is oriented to the inner side of the light outlet surface 16 with respect to its reception direction (compare FIG. 3). In this way, the return reflection of the laser radiation which is directed from the inner side of the light outlet surface 16 into the light-conducting fiber 5 impinges on the reception surface of the optoelectronic receiver 15 and can be evaluated in the latter.

In order to couple this return reflection out of the laser radiation 2 and deflect it in the direction of the optoelectronic receiver 15, an optical splitter 18 is arranged in the laser beam path 2 between the optical interrupter 8 and the input-coupling system 17 serving to couple the laser radiation into the light waveguides 5. If the intensity of the return reflection of the laser radiation 2 from the inner side of the light outlet surface 16 exceeds a predetermined threshold value, the optoelectronic receiver 15 triggers a signal which reaches the control electronics 10 via signal path 14 and, by means of the control electronics 10, causes the optical interrupter 8 to be switched on and accordingly causes the laser radiation 2 to be interrupted.

The intensity of the return reflection is influenced by the consistency of the material located at the treatment site. If the threshold value for the intensity at which the optoelectronic receiver 15 is to respond is set in such a way that it corresponds to the intensity of the return reflection when the material located at the treatment site passes into the gaseous state, the interruption of the laser radiation 2 is carried out in the manner described above at the same time that the material passes into this state or after a delay in time, as required. In this way, in case of an explosive evaporation of the aqueous material at the treatment site, i.e., directly in front of the light outlet surface 16, switching off the laser radiation 2 enables the cavitation chamber that has just been formed to fill with water and accordingly prevents excessively long shot channels and the consequences of such excessively long shot channels.

In a further arrangement for carrying out the method steps according to the invention (compare FIG. 3), the laser radiation 2 is directed from the mouth opening 19 of the suction tube 20 to the nucleus material to be comminuted. The eye lens 1 whose position with respect to the laser radiation follows from FIG. 1 and FIG. 2 is not shown in this case for the sake of clarity. In FIG. 3, the cross section of the light-conducting fiber 5 and the cross section of the suction tube 20 are round, for example, and the two cross sections are arranged eccentrically with respect to one another in such a way that the suction tube 20 has an opening which is arranged annularly around the light-conducting fiber 5 to allow the passage of ablation product (FIG. 4 as view A from FIG. 3). The mouth opening 19 is inclined by an angle β relative to the center axis 22 of the light-conducting fiber 5; the light outlet surface 16 is inclined relative to the center axis 22 by angle α.

The eccentric arrangement of the light-conducting fiber 5 relative to the cross section of the suction tube 20 (FIG. 4) results in the advantage that a relatively homogenized suction of the ablated material from the treatment site is carried out on the one hand, but the suction of larger ablation product is also possible without difficulty due to the relatively large distance a between the outer jacket of the light-conducting fiber 5 and the inner wall of the suction tube 20.

Alternatively, it can be provided by means of a similarly constructed arrangement that the light-conducting fiber 5 is arranged within the suction tube 20 so as to be rotatable about its center axis 27 in direction U and is connected with a rotary drive (not shown in the drawing).

As is shown in FIG. 3, the exiting direction of the laser radiation can be changed by manipulating the light outlet surface 16, especially by varying angle α. Accordingly, it is also clear that a rotation of the light-conducting fiber 5 about its center axis 22, for example, in direction U, results in a change in the radiation direction of the laser radiation 2 into the nucleus material. If a cavitation chamber is formed at the treatment site due to a rapid series of shots, a change in the radiation direction of the laser radiation 2 into the nucleus tissue is carried out by rotating the light-conducting fiber 5 in direction U and, in this way, the formation of a long shot channel is prevented. The cavitation chamber that has been formed and that now no longer lies in the target area of the laser radiation 2 can now be filled with water.

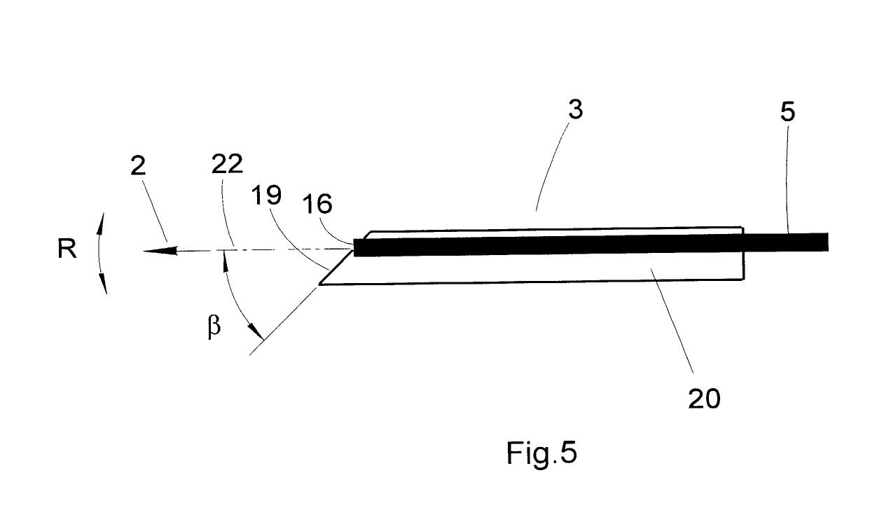

As an alternative to the constructional variant of this arrangement with a light-conducting fiber 5 which is rotatable about its center axis 22, it is possible for the light-conducting fiber 5 to be constructed such that it is not rotatable, but rather is constructed, with respect to the position of the light outlet surface 16, inside the mouth opening 19 through a deflection vertical to the radiation direction of the laser radiation 2 or to the center axis 22 (compare FIG. 5). In FIG. 5, the laser radiation 2 exits from a light outlet surface 16 which is oriented vertical to the center axis 22. For this reason, it is not deflected in this case; rather, its principal radiation direction lies in the direction of the center axis 22. Similarly to the procedure described above, the deflection in direction R can be initiated by means of an oscillating drive when there is a risk in the radiation direction of formation of a gas-filled cavitation chamber with consequent formation of a shot channel.

Manually entered actuating commands or signals which are obtained automatically, for example, such as those described above with reference to the returning reflection from the light outlet surface 16, can be utilized for the rotation of the light-conducting fiber 5 in direction U or also for changing the direction of the light-conducting fiber 5 in direction R.

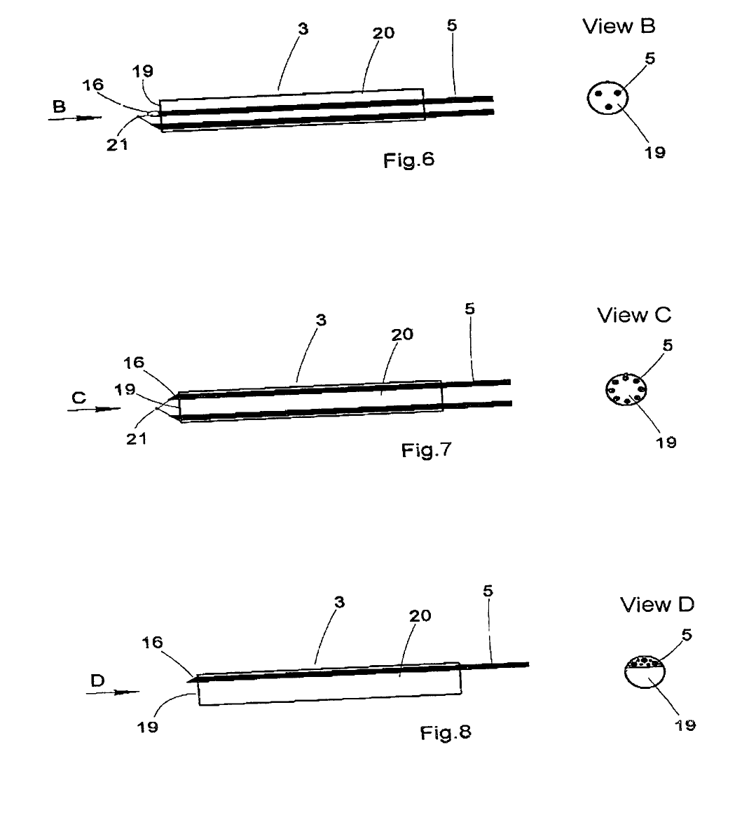

Further arrangements which prevent the risk of formation of excessively long shot channels are shown in FIG. 6, FIG. 7 and FIG. 8. In each case, a plurality of light-conducting fibers 5 are to be arranged in the suction tube 20 such that the laser radiation 2 is directed from the mouth opening 19 of the suction tube 20 to the treatment site.

In the arrangement according to FIG. 6, it is provided, for example, that the light outlet surfaces 16 of three light-conducting fibers 5 that are arranged so as to be radially symmetric to the center of the suction tube are inclined in such a way that the three light bundles are focussed on one point 21. If the intensity of the laser radiation transmitted by the three light-conducting fibers 5 is selected in such a way that ablation is possible only in the region of point 21, this also prevents the risk of formation of deeper shot channels because the beam bundles diverge again at a great distance from the light outlet openings 16 and the application of energy into the nucleus material is no longer sufficient to cause cavitation.

In FIG. 7, a plurality of light-conducting fibers 5 are distributed at the circumference of the suction tube 20. Accordingly, it is possible, for example, rather than radiating the laser light into the treatment site simultaneously through all of the light-conducting fibers 5 that are provided in this case, to alternate the transmission path for the laser radiation in that individual light-conducting fibers 5 or, as the case may be, a plurality of light-conducting fibers 5 are used in succession to transmit the laser radiation 2 from the laser 4 to the treatment site, so that the location of radiation into the tissue also changes at the treatment site in alternation with the transmission path.

In a manner similar to the configuration of the arrangement mentioned above, FIG. 8 shows a plurality of light-conducting fibers 5 which are not distributed along the circumference of the suction tube 20, but rather are arranged along a circular segment of the suction tube 20. By means of this arrangement, it is also possible to vary the radiation site for the laser radiation 2 in the tissue in that different light-conducting fibers 5 are acted upon in succession. This arrangement offers advantages with respect to the utilization of the cross-sectional surface because light-conducting fibers 5 of different diameters are provided in a surface-filling manner.

While the foregoing description and drawings represent the preferred embodiments of the present invention, it will be obvious to those skilled in the art that various changes and modifications may be made therein without departing from the true spirit and scope of the present invention.

What is claimed is:

1. A method for phacoemulsification in which the biological tissue of the lens of the eye is ablated comprising the steps of:
applying energy in the form of pulsed laser radiation to the lens of the eye for ablation of the lens of the eye;
varying the applied pulsed laser radiation by changing at least one of the pulse train and the intensity of the laser radiation; and
removing an occurring ablation product by suction from a treatment site.

2. The method according to claim 1, wherein the pulse train is changed in that an interruption of at least one pulse length is provided after every series of pulses at a constant pulse frequency.

3. The method according to claim 2, wherein a value in the range of 40 Hz to 100 Hz is provided for the constant pulse frequency and the ratio of the duration of a pulse series to an interruption is selected between 10:1 and 1:1.

4. The method according to claim 1, wherein the pulse train is influenced in that the pulse frequency of the laser radiation is alternately increased and reduced.

5. The method according to claim 1, wherein application of energy is varied depending on optical characteristics of the material at the treatment site.

6. The method according to claim 5, wherein return reflection of the laser radiation from a light outlet surface of a light waveguide serving to transmit the laser radiation to the treatment site is evaluated and the applied energy is changed depending on said return reflection.

7. The method for phacoemulsification of claim 1, wherein the radiating direction of the laser radiation in the tissue is varied depending on physical data of the tissue at the treatment site.

8. The method according to claim 7, wherein the radiation direction is varied depending on optical characteristics of the material at the treatment site.

9. The method according to claim 7, wherein the radiation direction is changed in dependence on the pressure at the treatment site.

10. An arrangement for phacoemulsification for comminution of the biological tissue of the eye lens comprising:
a laser for providing pulsed laser radiation for application to an eye lens;
a driving circuit for the laser;
at least one light waveguide for transmitting the laser radiation to a treatment site and radiating it into tissue at that location; and
means being provided for influencing at least one of the pulse train and the intensity of the laser radiation.

11. The arrangement according to claim 10, wherein said laser has an associated pulse train and laser beam path and a controllable optical interrupter is provided as a device for influencing the pulse train and is arranged in said laser beam path.

12. The arrangement according to claim 11, wherein the driving circuit for the laser communicates with a control input of an optical interrupter by control electronics; wherein the control electronics have a reference value input and the control electronics can be adjusted by said reference value input in such a way that a switching signal is supplied to the optical interrupter after every cycle of a number of pulses which is predetermined as a reference value, wherein the switching signal results in a blocking of the passage of the laser radiation for the duration of at least one pulse length.

13. The arrangement according to claim 12, wherein a reference value input of the control electronics is connected to the signal output of an optoelectronic receiver whose receiving direction is directed to the inside of the light outlet surface of the light waveguide.

14. An arrangement for phacoemulsification for a comminution of the biological tissue of the eye lens comprising:
a laser for providing pulsed laser radiation;
at least one light waveguide for transmitting the laser radiation to the treatment site;
a suction tube for removing occurring ablation product by suction through a mouth opening of the suction tube;
said light waveguide or light waveguides, insofar as more than one is provided, being arranged such that at least a portion thereof extends in the suction tube; and
said laser radiation being directed out of the mouth opening of the suction tube to the treatment site, wherein the end of the light waveguide on the light exit side is enclosed annularly by the suction tube cross section.

15. The arrangement according to claim 14, wherein only one light waveguide is provided, said light wavelength guide and said suction tube having associated cross sections, wherein the cross section of the light waveguide and the cross section of the suction tube are round, and both cross sections are arranged eccentrically relative to one another with respect to their center of curvature.

16. The arrangement according to claim 14, wherein a light outlet surface for the laser radiation is inclined relative to a center axis of the light waveguide by an angle a α≠90°.

17. The arrangement according to claim 14, wherein the mouth opening of the suction tube is inclined relative to the center axis of the light waveguide by an angle β≠90°.

18. The arrangement according to claim 14, wherein the light waveguide is arranged within the suction tube so as to be rotatable about its center axis and is connected with a rotary drive.

19. The arrangement according to claim 18, wherein the rotary drive has a driving circuit or control circuit which is linked with the signal output of an optoelectronic receiver whose reception surface is directed to the inner side of the light outlet surface of the light waveguide.

20. The arrangement according to claim 14, wherein a plurality of light waveguides is provided and wherein the light waveguides are coupled with at least one laser radiation source in such a way that different light waveguides are switched on alternately in succession for transmission of the laser radiation to the treatment site.
